(19) United States
(12) Patent Application Publication
(10) Pub. No.: US 2023/0068267 A1
(43) Pub. Date: Mar. 2, 2023

(54) PLANT EXTRACT COMPOSITION FOR THE TREATMENT OF CARDIOVASCULAR AND METABOLIC DISEASES

(71) Applicant: Meda Pharma S.p.A., Monza (IT)

(72) Inventors: Elena Gelfi, Monza (IT); Andrea Zanardi, Monza (IT); Franco Gasparri, Monza (IT)

(73) Assignee: Meda Pharma S.p.A., Monza (IT)

(21) Appl. No.: 17/789,646

(22) PCT Filed: Jan. 15, 2021
(86) PCT No.: PCT/EP2021/050853

§ 371 Date: Jun. 28, 2022

(30) Foreign Application Priority Data
Jan. 15, 2020 (GB) 2000619.3

(51) Int. Cl. A61K 31/7048; A61K 31/216; A61K 36/28; A61K 36/752; A61K 9/20; A61P 3/06; A61P 3/10; A61P 9/00; A61K 9/00

(52) U.S. Cl. CPC A61K 31/7048; A61K 31/216; A61K 36/28; A61K 36/752; A61K 9/2068; A61P 3/06; A61P 3/10; A61P 9/00; A61K 9/0053

(57) ABSTRACT

The invention relates to a combination of: naringin; and chlorogenic acid; for use in the treatment or prevention of dyslipidemia, cardiovascular diseases, metabolic syndrome and hypercholesterolemia. The invention also relates to compositions comprising the combination.

This invention relates to new compositions, in particular nutraceutical compositions, and their uses, in particular for treating or preventing dyslipidemia, cardiovascular diseases, metabolic syndrome and hypercholesterolemia.

### BACKGROUND OF THE INVENTION

Epidemiologic evidence implicates Western diets as a contributing factors in the development of cardiovascular diseases, dyslipidemia, and diabetes.

Fatty acids are vital components of many biological processes and are crucial in the pathogenesis of numerous common diseases. These molecules function both as an energy source and as signals for metabolic regulation, acting through enzymatic and transcriptional networks to modulate gene expression, growth and survival pathways, and inflammatory and metabolic responses.

However, recent evidence also suggests that a high-fat diet is responsible for the development of metabolic syndrome both in animals and in humans. Metabolic syndrome is a cluster of diseases, including hypertension, dyslipidemia, insulin-resistant diabetes and central (visceral) obesity. Metabolic syndrome is common and is associated with an increased risk for cardiovascular diseases (CVD) in both sexes.

Lifestyle and diet choices are important actions to control dyslipidemia and hypercholesterolemia. In particular, diet management can combine supplements with improved compliance with an appropriate dietary regimen. Previous studies suggest that the combining food supplements with diet leads to improved control of lipid metabolism.

However, often pharmaceutical or nutraceutical intervention is needed. Such agents have been successfully used for the treatment of major risk factors, including hypertension, plasma cholesterol, and hyperglycemia. Unfortunately, these agents generally cause adverse effects, such as coughs, dizziness, headaches, flushing, palpitations, angioedema, liver dysfunction and myositis.

Accordingly, it is an object of the present invention to provide further methods that can manage and treat these chronic diseases without causing the undesirable adverse effects.

### SUMMARY OF THE INVENTION

In a first aspect, the invention provides a combination of:
naringin; and
chlorogenic acid;
   for use in the treatment or prevention of dyslipidemia, cardiovascular diseases, metabolic syndrome and hypercholesterolemia.

The invention also provides a method for the treatment or prevention of dyslipidemia, cardiovascular diseases, metabolic syndrome and hypercholesterolemia comprising administering to a human a combination of:
naringin; and
chlorogenic acid,

The invention also provides the use of a combination of:
naringin; and
chlorogenic acid,
   for the manufacture of a medicament for the treatment or prevention of dyslipidemia, cardiovascular diseases, metabolic syndrome and hypercholesterolemia.

In a second aspect, the invention provides a combination of:
Citrus bergamia extract; and
Cynara cardunculus extract;
   for use in the treatment or prevention of dyslipidemia, cardiovascular diseases, metabolic syndrome and hypercholesterolemia.

The invention also provides a method for the treatment or prevention of dyslipidemia, cardiovascular diseases, metabolic syndrome and hypercholesterolemia comprising administering to a human a combination of:
Citrus bergamia extract; and
Cynara cardunculus extract.

The invention also provides the use of a combination of:
Citrus bergamia extract; and
Cynara cardunculus extract,
   for the manufacture of a medicament for the treatment or prevention of dyslipidemia, cardiovascular diseases, metabolic syndrome and hypercholesterolemia.

The combination of the invention is for use in the treatment or prevention of dyslipidemia, cardiovascular diseases, metabolic syndrome and hypercholesterolemia. Thus, the combination may be administered as a prophylactic treatment to prevent the condition developing, or to treat the condition after it has already developed.

Surprisingly, the applicant has found that the combination of the Citrus bergamia extract comprising naringin and the Cynara cardunculus extract comprising chlorogenic acid is useful for the treatment of dyslipidemia, cardiovascular diseases, metabolic syndrome and hypercholesterolemia by affecting mechanisms which lead to the accumulation of cholesterol in the body.

### BRIEF DESCRIPTION OF THE DRAWINGS

Additional aspects of the invention are more fully described in the following detailed description of the various embodiments with reference to the accompanying drawings, in which:

FIG. 1. shows the viability of HepG2 liver cells previously exposed to a 2.5 μM mixture of free fatty acids after being treated with 1, 5, 10 and 15 μg/mL solutions of Citrus bergamia extract containing naringin;

FIG. 2. shows the viability of HepG2 liver cells previously exposed to a 3.0 μM mixture of free fatty acids after being treated with 1, 5, 10 and 15 μg/mL solutions of Citrus bergamia extract containing naringin;

FIG. 3. shows the viability of HepG2 liver cells previously exposed to a 2.5 μM mixture of free fatty acids after being treated with 1, 5, 10 and 15 μg/mL solutions of Cynara cardunculus extract containing chlorogenic acid;

FIG. 4. shows the viability of HepG2 liver cells previously exposed to a 3.0 μM mixture of free fatty acids after being treated with 1, 5, 10 and 15 μg/mL solutions of Cynara cardunculus extract containing chlorogenic acid;

FIG. 5. shows the viability of HepG2 liver cells previously exposed to a 2.5 μM mixture of free fatty acids after being treated with a mixture of 1 μg/mL Citrus bergamia extract containing naringin and 15 μg/mL Cynara cardunculus extract containing chlorogenic acid; and

FIG. 6. shows the viability of HepG2 liver cells previously exposed to a 3.0 μM mixture of free fatty acids after being treated with a mixture of 1 μg/mL Citrus bergamia extract containing naringin and 15 μg/mL Cynara cardunculus extract containing chlorogenic acid.

### DEFINITIONS

The proportions of the various components of the combination are defined relative to other components. The wt % (weight percent) of a particular component, based on the other components, is the weight (mass) of the particular component, divided by the weight (mass) of the other components, times by 100 i.e.

$$wt \% single component X ( based on weight of the botanical extract Y ) = \\ \frac{wt ( X )}{wt ( Y )} ⨯ 100$$

Cynara cardunculus belongs to Asteracea botanical family. Cynara cardunculus extract includes the taxa/species such as: Cynara cardunculus L. var. sylvetris lam., Cynara cardunculus L. var. altis DC, C. cardunculus subsp. scolymus (L.) hegi, Cynara cardunculus L. var.scolymus (L.)fiori (also named Cynara scolymus L.) The plant is cultivated in Europe, and the harvest period is from April to October. The extracts are collected from the leaves of the plant.

Bergamot, the common name of Citrus bergamia risso, belongs to the family Rutaceae, subfamily Esperidea and it has been widespread in the Mediterranean area for centuries. The tree Citrus bergamia is found in the Calabria region specifically, due to its unique climate that is suitable for its growth.

### DETAILED DESCRIPTION OF THE INVENTION

In a first aspect, the invention provides a combination of:
naringin; and
chlorogenic acid;
   for use in the treatment or prevention of dyslipidemia, cardiovascular diseases, metabolic syndrome and hypercholesterolemia.

The invention also provides naringin for use in the treatment or prevention of dyslipidemia, cardiovascular diseases, metabolic syndrome and hypercholesterolemia, wherein the naringin is administered in combination with chlorogenic acid.

The invention also provides chlorogenic acid for use in the treatment or prevention of dyslipidemia, cardiovascular diseases, metabolic syndrome and hypercholesterolemia, wherein the chlorogenic acid is administered in combination with naringin.

In a second aspect, the invention provides a combination of:
Citrus bergamia extract; and
Cynara cardunculus extract;
   for use in the treatment or prevention of dyslipidemia, cardiovascular diseases, metabolic syndrome and hypercholesterolemia.

The invention also provides Citrus bergamia extract for use in the treatment or prevention of dyslipidemia, cardiovascular diseases, metabolic syndrome and hypercholesterolemia, wherein the Citrus bergamia extract is administered in combination with Cynara cardunculus extract.

The invention also provides Cynara cardunculus extract for use in the treatment or prevention of dyslipidemia, cardiovascular diseases, metabolic syndrome and hypercholesterolemia, wherein the Cynara cardunculus extract is administered in combination with Citrus bergamia extract.

The applicant has also found that the combination of naringin and chlorogenic acid, or the combination of Citrus bergamia extract comprising naringin and the Cynara cardunculus extract comprising chlorogenic acid is useful for the treatment or prevention of dyslipidemia, particularly hypercholesterolemia.

That is, the combinations of the invention may be used to treat or prevent dyslipidemia, preferably mixed dyslipidemia (hypercholesterolemia and hypertriglyceridemia), hypercholesterolemia, preferably familial or polygenic, hypertriglyceridemia, diabetes associated with dyslipidemia, statin-induced myalgia or myopathy, intolerance to hypolipidemising drugs, clinical conditions characterized by low HDL cholesterol levels, or atherosclerosis.

Preferably, the combination is for use in the treatment or prevention of dyslipidemia, preferably mixed dyslipidemia (hypercholesterolemia and hypertriglyceridemia), hypercholesterolemia, preferably familial or polygenic, hypertriglyceridemia, diabetes associated with dyslipidemia.

### Naringin

Naringin is bitter-tasting flavanone-7-O-glycoside between the flavanone naringenin and the disaccharide neohesperidose. It has the chemical name 7-[[2-O-(6-Deoxy-α-L-mannopyranosyl)-β-D-glucopyranosyl]oxy]-2,3-dihydro-5-hydroxy-2-(4-hydromphenyl)-4H-1-benzopyran-4-one, and has the following structure:

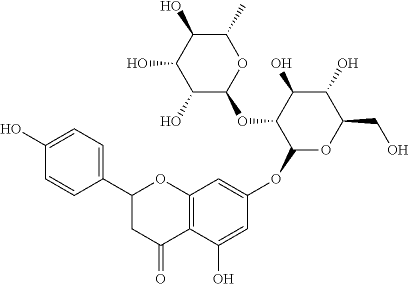

Naringin can be extracted from Citrus bergamia, Citrus paradisi, Citrus sulcata, Citrus aurantium, Citrus sinensis or Citrus erythrosa (see M. Yano et. al., J. Agric Food Chem 1999, 47, 128-135; Tables 1 and 2).

### Chlorogenic acid

Chlorogenic acid is the ester of caffeic acid and (-)-quinic acid. It has the chemical name (1S,3R,4R,5R)-3-{[(2E)-3-(3,4-dihydroxyphenyl)prop-2-enoyl]oxy}-1,4,5-trihydroxycyclohexanecarboxylic acid, and has the following structure:

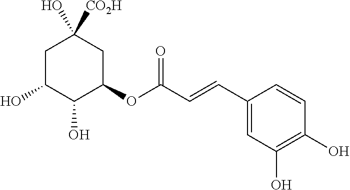 [SMILES: O=C(/C=C/c1ccc(O)c(O)c1)O[C@@H]1C[C@](O)(C(=O)O)C[C@@H](O)[C@H]1O]

Chlorogenic acid can also be extracted from Cynara cardunculus i.e. the artichoke, by methods known in the art.

### Neoeriocitrin and Neohesperidin

Neoeriocitrin is a 7-O-glycoside of the flavanone eriodictyol and the disaccharide neohesperidose. It has the chemical name (S)-3′,4′,5,7-Tetrahydroxyflavanone-7-[2-O-(α-L-rhamnopyranosyl)-β-D-glucopyranoside], and has the following structure:

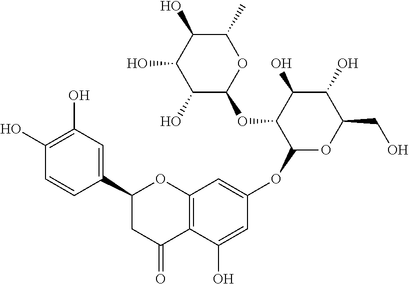

Neohesperidin is the 7-O-neohesperidose derivative of hesperetin, and has the following structure:

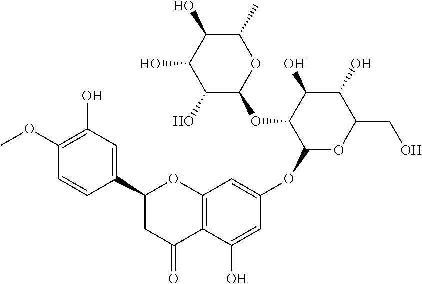

Neoeriocitrin and neohesperidin can also be extracted from Citrus bergamia, Citrus paradisi, Citrus sulcata, Citrus aurantium, Citrus sinensis or Citrus erythrosa (see M. Yano et. al., J. Agric Food Chem 1999, 47, 128-135; Tables 1 and 2). Neoeriocitrin and neohesperidin may be present in various embodiments, in particular in combinations used in the present invention.

### Melitidin and Brutieridin

Melitidin and brutieridin are flavanone glycosides and have the following structure:

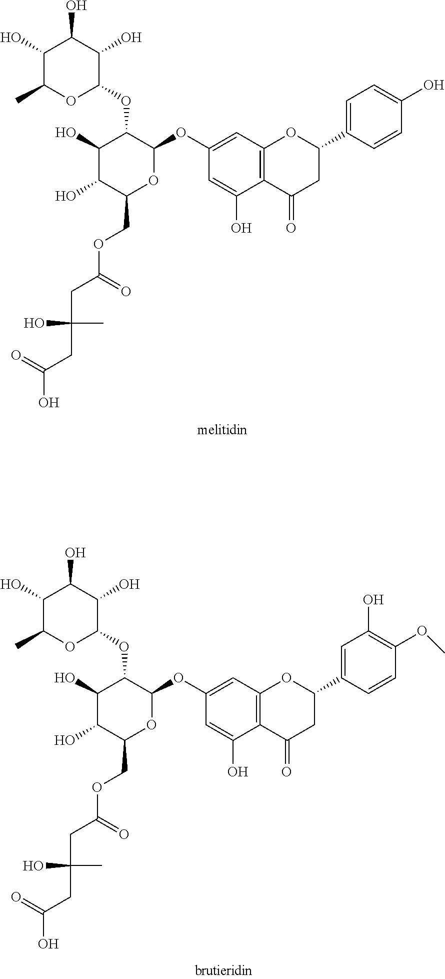

Melitidin and brutieridin can also be extracted from Citrus bergamia. Both compounds have statin-like properties owing to their inhibitory action upon HMG-CoA reductase (Di Donna 2009). Melitidin and brutieridin may also be present in various embodiments, in particular in combinations used in the present invention.

### Rutin

Rutin is a citrus flavonoid and has the following structure:

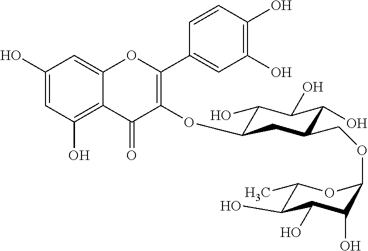

Rutin can be extracted from a wide variety of plants, including Citrus bergamia. Rutin is known to inhibit the oxidation of LDL cholesterol (Yu et. al. 2005). Rutin may also be present in various embodiments, in particular in combinations used in the present invention.

The combination

In one embodiment, the combination used in the present invention includes:
naringin; and
chlorogenic acid;

In one embodiment, the wt ratio of naringin to chlorogenic acid is selected from
10:1 to 1:10;
9:1 to 1:9;
8:1 to 1:8;
7:1 to 1:7;
6:1 to 1:6;
5:1 to 1:5;
4:1 to 1:4;
3:1 to 1:3; and
2:1 to 1:2.

In one embodiment, the wt ratio of naringin to chlorogenic acid is selected from
1:1 to 1:10;
1:1 to 1:9;
1:1 to 1:8;
1:1 to 1:7;
1:1 to 1:6;
1:1 to 1:5;
1:1 to 1:4;
1:1 to 1:3; and
1:1 to 1:2.

In one embodiment, the wt ratio of naringin to chlorogenic acid is selected from
10:1 to 1:1;
9:1 to 1:1;
8:1 to 1:1;
7:1 to 1:1;
6:1 to 1:1;
5:1 to 1:1;
4:1 to 1:1;
3:1 to 1:1; and
2:1 to 1:1.

In one embodiment, the combination used in the present invention includes:
naringin;
neohesperidin; and
chlorogenic acid.

In one embodiment, the combination used in the present invention includes:
naringin;
neoeriocitrin; and
chlorogenic acid

In one embodiment, the combination used in the present invention includes:
naringin;
neoeriocitrin;
neohesperidin; and
chlorogenic acid.

In one embodiment, the combination used in the present invention includes:
naringin;
neoeriocitrin;
neohesperidin;
melitidin;
brutieridin;
rutin; and
chlorogenic acid.

In another embodiment, the combination used in the present invention includes:
Citrus bergamia extract; and
Cynara cardunculus extract.

In another embodiment, the combination used in the present invention includes:
Citrus bergamia extract comprising naringin; and
Cynara cardunculus extract.

In another embodiment, the combination used in the present invention includes:
Citrus bergamia extract; and
Cynara cardunculus extract comprising chlorogenic acid.

In another embodiment, the combination used in the present invention includes:
Citrus bergamia extract comprising naringin; and
Cynara cardunculus extract comprising chlorogenic acid.

In another embodiment, the combination used in the present invention includes:
Citrus bergamia extract comprising naringin, neoeriocitrin, and neohesperidin; and
Cynara cardunculus extract comprising chlorogenic acid.

In another embodiment, the combination used in the present invention includes:
Citrus bergamia extract comprising naringin, neoeriocitrin, and neohesperidin, melitidin, brutieridin, rutin; and
Cynara cardunculus extract comprising chlorogenic acid.

In another embodiment, the combination used in the present invention includes:
Citrus bergamia extract; and
Cynara cardunculus extract.

In one embodiment, the wt ratio of the Citrus bergamia extract to the Cynara cardunculus extract is selected from:
10:1 to 1:10;
9:1 to 1:9;
8:1 to 1:8;
7:1 to 1:7;
6:1 to 1:6;
5:1 to 1:5;
4:1 to 1:4;
3:1 to 1:3; and
2:1 to 1:2.

In one embodiment, the wt ratio of the Citrus bergamia extract to the Cynara cardunculus extract is selected from:
1:1 to 1:10;
1:1 to 1:9;
1:1 to 1:8;
1:1 to 1:7;
1:1 to 1:6;
1:1 to 1:5;
1:1 to 1:4;
1:1 to 1:3; and
1:1 to 1:2.

In one embodiment, the wt ratio of the Citrus bergamia extract to the Cynara cardunculus extract is selected from:
10:1 to 1:1;
9:1 to 1:1;
8:1 to 1:1;
7:1 to 1:1;
6:1 to 1:1;
5:1 to 1:1;
4:1 to 1:1;
3:1 to 1:1; and
2:1 to 1:1.

In one embodiment of the invention, the combination does not include administration of L-ascorbic acid i.e. the patient to whom the components of the combination is not also being administered L-ascorbic acid.

In one embodiment, the Citrus bergamia extract comprises from 30% to 70% w/w flavonoids, preferably 35% to 65% w/w e.g. 40% w/w.

In one embodiment, the Citrus bergamia extract comprises from 5% to 25% w/w naringin, preferably 10% to 20% w/w e.g. 15% w/w.

In one embodiment, the Citrus bergamia extract comprises from 2.5% to 20% w/w neoeritrocitrin, preferably 5% to 15% w/w e.g. 10% w/w.

In one embodiment, the Citrus bergamia extract comprises from 5% to 25% w/w neohesperidin, preferably 10% to 20% w/w e.g. 15% w/w.

In one embodiment, the Citrus bergamia extract comprises from 0.5% to 5% w/w melitidin, preferably 1% to 4% w/w e.g. 3% w/w.

In one embodiment, the Citrus bergamia extract comprises from 1% to 7% w/w brutieridin, preferably 2% to 6% w/w e.g. 5% w/w.

In one embodiment, the Citrus bergamia extract comprises from 0.1% to 0.5% w/w rutin, preferably 0.2% to 0.4% w/w e.g. 0.2% w/w.

In one embodiment, the Citrus bergamia extract comprises:
from 10% to 20% w/w naringin;
from 5% to 15% w/w neoeritrocitrin; and
from 10% to 20% w/w neohesperidin.

In one embodiment, the Citrus bergamia extract comprises:
from 10% to 20% w/w naringin;
from 5% to 15% w/w neoeritrocitrin;
from 10% to 20% w/w neohesperidin;
from 0.5% to 5% w/w melitidin;
from 1% to 7% w/w brutieridin: and
from 0.1% to 0.5% w/w rutin.

In one embodiment, the Cynara cardunculus extract comprises from 1% to 5% w/w flavonoids, preferably 1% to 3% w/w e.g.1.5% w/w.

In one embodiment, the Cynara cardunculus extract comprises from 1% to 10% w/w chlorogenic acid, preferably 3% to 8% w/w e.g. 5% w/w.

In one embodiment, the Cynara cardunculus extract comprises from 1% to 10% w/w caffeoylquinic acids, preferably 3% to 10% w/w e.g. 6% w/w.

In one embodiment, the Cynara cardunculus extract comprises:
from 3% to 10% w/w chlorogenic acid; and
from 3% to 10% w/w caffeoylquinic acids.

### Preparation of the Extract

In one embodiment, the Citrus bergamia extract is obtainable by chromatographic absorption followed by desorption using a solvent (e.g. water:ethanol 1:1). The bergamot juice is first microfiltered and then extracted by adsorption chromatography. The resins of the columns are washed with a solution of ethanol and water.

The resulting liquid is then concentrated at 40° C. under vacuum, and then combined with maltodextrin and silica. The resulting liquid is then subjected to a spray drying step and milled. The final homogenization takes place through a double conic blender and filling into a drum.

The chromatographic adsorption/microfiltration used to obtain the Citrus bergamia extract provides an extract with a high flavonoid content (40% w/w) which is particularly advantageous. The physical adsorption technique and the use of columns with a high number of theoretical plates make it possible to achieve a concentration of flavonoids that cannot be accessed with other known extraction techniques.

The following concentrations of extracts can be obtained depending on the technique used:

| | Extract (%) | | | | | | |
|---|---|---|---|---|---|---|---|
| Extraction | | | | | | | Total |
| technique | Naringin | Neoeriocitrin | Neohesperidin | Melitidin | Brutieridin | Rutin | flavonoids |
| Solvent: water | 4 | 3 | 3 | — | — | — | 10 |
| Solvent: water | 7 | 6 | 8 | — | — | — | 21 |
| and alcohols | | | | | | | |
| Ultrafiltration | 9 | 7 | 8 | 1 | 2 | — | 27 |
| and clarification | | | | | | | |
| Adsorption | 16 | 9 | 15 | 3 | 5 | 1 | 49 |
| chromatography | | | | | | | |

In one embodiment, the Cynara cardunculus extract is obtainable by conventional solvent extraction for example using water:ethanol e.g. 1:3.

Prior to performing the solvent extraction several steps may be performed. For example, the leaves of an artichoke are collected and dried, typically at a temperature between 40° and 50° C. Once dry, the leaves are subjected to a milling step to reduce their size. The cut leaves are then subjected to a reverse flow solid/liquid extraction with water/ethanol (1:3) at 40° C. The resulting extracts are then filtered through a membrane and centrifuged. Following centrifugation, the extracts are concentrated under reduced pressure and are then subjected to a liquid/liquid extraction with ethyl acetate. The resulting extracts are then separated by high speed centrifugation (900 rpm) and then concentrated under reduced pressure. The concentrate is then dried and homogenised using a trough cutter miller.

The solid/liquid high-temperature extraction technique used, in which the dry extract of artichoke leaves comes into contact with the extractive substance through successive steps enriched with functional ingredients, makes it possible to obtain an extract particularly rich in chlorogenic acid and caffeoylquinic acids.

### The Use in Combination

The combinations of the invention may produce an increased therapeutic effect relative to the therapeutic effect of the individual components when administered alone.

In particular, the combination may, relative to the individual components when administered alone, provide additivity and synergism.

A “synergistic” effect occurs when the combination provides an effect which is larger than the sum of the therapeutic effects of the agents administered alone.

An “additive” effect occurs when the combination provides an effect which is larger than the either of the components when administered alone.

The term “combination” means that the components are administered as part of the same overall treatment regimen.

The components may be administered at the same time or at different times. It will therefore be appreciated that the components of the combination may be administered sequentially (e.g. before or after) or simultaneously, either in the same formulation (i.e. together), or in different formulations (i.e. separately).

In one embodiment, the components are administered simultaneously in the same formulation i.e. a unitary formulation comprising all components in the same dose.

In one embodiment, the components are administered simultaneously in different formulations. In one embodiment, the components are administered separately or sequentially in different formulations.

### Compositions

In a third aspect, the invention provides a pharmaceutical or nutraceutical composition comprising a combination of:
naringin; and
chlorogenic acid;
   and a pharmaceutically or nutraceutically acceptable excipient, wherein the composition does not include L-ascorbic acid.

In a fourth aspect, the invention provides a pharmaceutical or nutraceutical composition comprising a combination of:
Citrus bergamia extract; and
Cynara cardunculus extract;
   and a pharmaceutically or nutraceutically acceptable excipient, wherein the composition does not include L-ascorbic acid.

In a fifth aspect the invention provides a pharmaceutical or nutraceutical composition comprising a combination of:
naringin; and
chlorogenic acid;
   and a pharmaceutically or nutraceutically acceptable excipient, wherein the naringin and the chlorogenic acid are present at from 0.1 to 5000 mg, or 1 to 1500 mg, 2 to 800 mg, or 5 to 500 mg, e.g. 2 to 200 mg or 10 to 1000 mg.

In an sixth aspect the invention provides a pharmaceutical or nutraceutical composition comprising a combination of:
Citrus bergamia extract; and
Cynara cardunculus extract;
   and a pharmaceutically or nutraceutically acceptable excipient, wherein the Citrus bergamia extract and the Cynara cardunculus extract are present at from 0.1 to 5000 mg, or 1 to 1500 mg, 2 to 800 mg, or 5 to 500 mg, e.g. 2 to 200 mg or 10 to 1000 mg.

The invention provides a pharmaceutical or nutraceutical composition comprising a combination of:
naringin; and
chlorogenic acid;
   and a pharmaceutically or nutraceutically acceptable excipient, wherein the weight ratio of naringin to chlorogenic acid is selected from:
10:1 to 1:10;
9:1 to 1:9;
8:1 to 1:8;
7:1 to 1:7;
6:1 to 1:6;
5:1 to 1:5;
4:1 to 1:4;
3:1 to 1:3; and
2:1 to 1:2.

The invention provides a pharmaceutical or nutraceutical composition comprising a combination of:
naringin; and
chlorogenic acid;
   and a pharmaceutically or nutraceutically acceptable excipient, wherein the weight ratio of naringin to chlorogenic acid is selected from:
1:1 to 1:10;
1:1 to 1:9;
1:1 to 1:8;
1:1 to 1:7;
1:1 to 1:6;
1:1 to 1:5;
1:1 to 1:4;
1:1 to 1:3; and
1:1 to 1:2.

The invention provides a pharmaceutical or nutraceutical composition comprising a combination of:
naringin; and
chlorogenic acid;
   and a pharmaceutically or nutraceutically acceptable excipient, wherein the weight ratio of naringin to chlorogenic acid is selected from:
10:1 to 1:1;
9:1 to 1:1;
8:1 to 1:1;
7:1 to 1:1;
6:1 to 1:1;
5:1 to 1:1;
4:1 to 1:1;
3:1to 1:1;and
2:1 to 1:1.

The invention provides a pharmaceutical or nutraceutical composition comprising a combination of:
Citrus bergamia extract; and
Cynara cardunculus extract;
   and a pharmaceutically or nutraceutically acceptable excipient, wherein the weight ratio of Citrus bergamia extract to Cynara cardunculus extract is selected from:
10:1 to 1:10;
9:1 to 1:9;
8:1 to 1:8;
7:1 to 1:7;
6:1 to 1:6;
5:1 to 1:5;
4:1 to 1:4;
3:1 to 1:3; and
2:1 to 1:2.

The invention provides a pharmaceutical or nutraceutical composition comprising a combination of:
Citrus bergamia extract; and
Cynara cardunculus extract;
   and a pharmaceutically or nutraceutically acceptable excipient, wherein the weight ratio of Citrus bergamia extract to Cynara cardunculus extract is selected from:
1:1 to 1:10;
1:1 to 1:9;
1:1 to 1:8;
1:1 to 1:7;
1:1 to 1:6;
1:1 to 1:5;
1:1 to 1:4;
1:1 to 1:3; and
1:1 to 1:2.

The invention provides a pharmaceutical or nutraceutical composition comprising a combination of:
Citrus bergamia extract; and
Cynara cardunculus extract;
   and a pharmaceutically or nutraceutically acceptable excipient, wherein the weight ratio of Citrus bergamia extract to Cynara cardunculus extract is selected from:
10:1 to 1:1;
9:1 to 1:1;
8:1 to 1:1;
7:1 to 1:1;
6:1 to 1:1;
5:1 to 1:1;
4:1 to 1:1;
3:1 to 1:1; and
2:1 to 1:1.

### Dosage

The combinations of the invention are useful in the treatment or prevention dyslipidemia, cardiovascular diseases, metabolic syndrome and hypercholesterolemia. The combinations of the invention are useful in the treatment or prevention of dyslipidemia, preferably mixed dyslipidemia (hypercholesterolemia and hypertriglyceridemia), hypercholesterolemia, preferably familial or polygenic, hypertriglyceridemia, diabetes associated with dyslipidemia, statin-induced myalgia or myopathy, intolerance to hypolipidemising drugs, clinical conditions characterized by low HDL cholesterol levels, or atherosclerosis.

The combinations of the invention are useful in the treatment or prevention of cardiovascular disease or metabolic syndrome.

The combinations of the invention are useful in the treatment or prevention of hypercholesterolemia.

The combination is generally administered to a subject in need of such administration, for example a human or animal, typically a human.

The combination will typically be administered in amounts that are therapeutically or prophylactically useful.

The compounds may be administered over a prolonged term to maintain beneficial therapeutic effects or may be administered for a short period only.

A typical daily dose of each components of the combination can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required.

The components of the combination may be administered orally in a range of doses, for example 0.1 to 5000 mg, or 1 to 1500 mg, 2 to 800 mg, or 5 to 500 mg, e.g. 2 to 200 mg or 10 to 1000 mg. Particular examples of doses including 10, 20, 50 and 80 mg.

In one embodiment, the pharmaceutical or nutraceutical composition comprises from 150 to 500 mg Citrus bergamia extract, preferably 300 to 400 mg.

In one embodiment, the pharmaceutical or nutraceutical composition comprises from 30 mg to 90 mg naringin, preferably 35 mg to 50 mg e.g. 40 mg.

In one embodiment, the pharmaceutical or nutraceutical composition comprises from 10 mg to 40 mg neoeritrocitrin, preferably 15 mg to 35 mg e.g. 25 mg.

In one embodiment, the pharmaceutical or nutraceutical composition comprises from 20 mg to 90 mg neohesperidin, preferably 30 mg to 80 mg e.g. 35 mg.

In one embodiment, the pharmaceutical or nutraceutical composition comprises from 1 mg to 25 mg melitidin, preferably 4 mg to 18 mg.

In one embodiment, the pharmaceutical or nutraceutical composition comprises from 1 mg to 35 mg brutieridin, preferably 6 mg to 30 mg.

In one embodiment, the pharmaceutical or nutraceutical composition comprises from 0.1 mg to 1 mg rutin, preferably 0.2 mg to 0.6 mg.

In one embodiment, the pharmaceutical or nutraceutical composition comprises:
from 30 mg to 90 mg naringin;
from 10 mg to 40 mg neoeritrocitrin; and
from 20 mg to 90 mg neohesperidin.

In one embodiment, the pharmaceutical or nutraceutical composition comprises:
from 30 mg to 90 mg naringin;
from 10 mg to 40 mg neoeritrocitrin;
from 20 mg to 90 mg neohesperidin;
from 4 mg to 18 mg melitidin;
from 6 mg to 30 mg brutieridin; and
from 0.1 mg to 1 mg rutin.

In one embodiment, the pharmaceutical or nutraceutical composition comprises from 80 mg to 800 mg Cynara cardunculus extract, preferably 100 to 700 mg.

In one embodiment, the pharmaceutical or nutraceutical composition comprises from 1 mg to 50 mg chlorogenic acid, preferably 3 mg to 35 mg e.g. 25 mg.

In one embodiment, the pharmaceutical or nutraceutical composition comprises:
from 1 mg to 50 mg chlorogenic acid; and
from 30 mg to 90 mg naringin.

### Formulations

In one embodiment, one or more of the components of the combination are provided as oral dosage forms. Oral dosage forms include tablets (coated or uncoated), capsules (hard or soft shell), caplets, pills, lozenges, syrups, solutions, powders, granules, elixirs and suspensions, sublingual tablets, wafers or patches such as buccal patches. Oral dosage forms may also include sachets or stick packs.

Preferably, the compositions of the invention are provided as tablets.

Therefore, in one embodiment of the invention, at least one of the components (preferably all of the components) is presented in a tablet. In one embodiment, all of the components are presented in tablets, and in particular all components of the combination are presented in the same tablet i.e. the combination is administered in a unitary or fixed-dose.

Typically, the tablet includes one or more pharmaceutically or nutraceutically acceptable excipient. The pharmaceutically or nutraceutically acceptable excipient can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavouring agents, sweeteners, taste masking agents, stabilisers or any other excipients conventionally used in pharmaceutical or nutraceutical compositions.

Preferably, the compositions of the invention are formulated with a pharmaceutically acceptable filler or bulking agent.

Examples of excipients include dibasic calcium phosphate anhydrous, magnesium stearate, silicon dioxide, carboxymethylcellulose, crospovidone, hydroxypropyl cellulose and maltodextrin.

Preferably, the compositions of the invention are provided in capsules.

Therefore, in one embodiment of the invention, at least one of the components (preferably all of the components) is presented in a capsule. In one embodiment, all of the components are presented in capsules, and in particular all components of the combination are presented in the same capsule i.e. the combination is administered in a unitary or fixed-dose.

Typically, the capsule includes one or more pharmaceutically or nutraceutically acceptable excipient. The pharmaceutically or nutraceutically acceptable excipient can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavouring agents, sweeteners, taste masking agents, stabilisers or any other excipients conventionally used in pharmaceutical compositions.

Examples of excipients include dibasic calcium phosphate anhydrous, magnesium stearate, silicon dioxide, maltodextrin, carboxymethylcellulose, crospovidone, and hydroxypropyl cellulose.

Preferably, the compositions of the invention are provided as granulates.

Therefore, in one embodiment of the invention, at least one of the components (preferably all of the components) is presented as a granulate. In one embodiment, all of the components are presented in a granulate, and in particular all components of the combination are presented in a single granulate i.e. the combination is administered in a unitary or fixed-dose. The granulate may be packaged into a sachet or a stick pack.

The granulate may be prepared by dry or wet granulation techniques that are known in the art.

### EXAMPLES

### Example 1: Synthesis

Citrus Bergamia Extract

Preparation

Bergamot (Citrus bergamia risso & poiteau) is a citrus fruit grown substantially only in restricted areas of Calabria and Sicily. The harvest period is from October to December. The Bergamot fruits are manually collected.

Extraction system for Bergamot is made with adsorption chromatography after a microfiltration. The final extract below is made with water/ethanol 1:1. More specifically, in order to obtain the extracts, the bergamot fruit was washed, and the juice was obtained using an FMC or JBT citrus juice extraction system. The juice was then subjected to a filtration, extraction and concentration process. The juice was filtered (filtration membranes with a pore size range of 0.05-2.0 microns and filtration pressure in the range of 0.5-2 bar), and then the filtrate was adsorbed onto a chromatographic resin column with a high number of theoretical plates. Following adsorption, the column was eluted with solvent (water/ethanol 1:1). The fractions were collected and concentrated under reduced pressure at 40° C. The concentrate was then dissolved in water to provide an aqueous solution, to which maltodextrin 19-25% (w/w) was added. The resulting solution was then subjected to a spray drying step (inlet temperature 180-185° C., outlet temperature 90° C.) to obtain a solid extract. Final drug extract ratio for Citrus bergamia is 214:1, that is 214 parts Citrus bergamia juice to 1 part drug extract.

The Citrus bergamia extract included the following components:

| | Concentration of |
|---|---|
| Component | component (w/w %) |
| Naringin | 15 ± 5 |
| Neoeritrocitrin | 10 ± 5 |
| Neohesperidin | 15 ± 5 |

A further Citrus bergamia extract included the following components:

| | Concentration of |
|---|---|
| Component | component (w/w %) |
| Naringin | 15 ± 5 |
| Neoeritrocitrin | 10 ± 5 |
| Neohesperidin | 15 ± 5 |
| Melitidin | 3 ± 2 |
| Bru tieridin | 5 ± 2 |
| Rutin | 0.2 ± 0.1 |

Aqueous Solutions

Aqueous solutions comprising Citrus bergamia extract were prepared by adding the extract to water at the following concentrations:

| | Concentration of |
|---|---|
| Label | extract (μg/mL) |
| C1 | 1 |
| C5 | 5 |
| C10 | 10 |
| C15 | 15 |

Cynara Cardunculus Extract

Preparation

The extracts may be collected by extraction from Cynara cardunculus species described herein with water/ethanol e.g. 1:3, concentration, liquid extraction with Ethyl acetate and drying. More specifically, in order to obtain the extracts, the leaves of the plant are collected and subjected to the following process.

Cynara cardunculus leaves were collected and dried at a temperature between 40° and 50° C. The drying temperature is constantly within this range, however the drying time may depend on the drying apparatus that is used. For example, the leaves may be dried at a temperature between 40° and 50° C. in a static drier for 48 hours, or the leaves may be dried at a temperature between 40° and 50° C. in a belt drier for 9 hours.

Once dry, the leaves were subjected to a milling step in order to reduce their size. A milling apparatus was fitted with a sieve of 1 cm diameter to ensure that the leaves were appropriately cut to size. The cut leaves were then subjected to a reverse flow solid/liquid extraction with water/ethanol (1:3) at 40° C. The resulting extracts were then filtered (filtration membranes with a pore size range of 0.05-2.0 microns and filtration pressure in the range of 0.5-2 bar) and centrifuged. Following centrifugation, the extracts were concentrated under reduced pressure at 40° C. and the concentrate was subjected to a liquid/liquid extraction with ethyl acetate. The resulting extracts were then separated by high speed centrifugation (900 rpm) and concentrated under reduced pressure at 40° C. The concentrate was then dried in a microwave desiccator (20 mBar at 30-35° C.) and homogenised using a trough cutter miller.

The homogenised extract was then mixed and standardised with dehydrated glucose syrup (from corn) cardunculus dry extract 80%; syrup corn dehydrated 20%.

The Cynara cardunculus extract included the following components:

| | Concentration of |
|---|---|
| Component | component (w/w %) |
| Chlorogenic acid | 5 ± 2 |
| Caffeoylquinic acids | 6 ± 2 |

Aqueous Solutions

Aqueous solutions comprising Cynara cardunculus extract were prepared by adding the extract to water at the following concentrations:

| | Concentration of |
|---|---|
| Label | extract (μg/mL) |
| D1 | 1 |
| D5 | 5 |
| D10 | 10 |
| D15 | 15 |

FFA Formulation

A solution of free fatty acids (FFAs) comprising oleic and palmitic acid at a ratio 2:1 was prepared in two concentrations:

| | Concentration |
|---|---|
| Label | of FFA (mM) |
| FFA 2.5 | 2.5 |
| FFA 3 | 3.0 |

### Example 2: Hepatoprotection

Assay Protocol

The assay protocol that was employed is described in detail by M. J. Gomez-Lechon et. al., in “A human hepatocellular in vitro model to investigate steatosis” Chemico-Biological Interactions 2007, 106-116.

The experiments were conducted on a human hepatocyte-derived cell line of immortalised hepatocytes called HepG2. The cells were kept in DMEM (Dulbecco's Modified Eagle Medium) culture medium (Gibco, BRL, Germany), added with 10% fetal bovine serum (FBS) (Gibco, BRL, Germany), 0.5% of gentamicin (Gibco, BRL, Germany) and 1% glutamine (Gibco, BRL, Germany).

The experiments on the xCELLigence® platform were performed using the RTCA DP (Dual Plate) using a validated setting. The platform was arranged to include three different components: (i)RTCA DP analyser, located within an incubator to maintain the cell cultures at 37° C. and 5% CO₂; (ii) RTCA control unit with RTCA software; and (ii) E-Plate 16 for seeding HepG2 cells.

The cells were divided into three groups: an untreated group (control), and groups treated for 24 hours either FFA 2.5 mM or FFA 3.0 mM.

Cell Index Measurement

The treated cell cultures were subsequently left untreated or were treated with C1, C5, C10, C15, D1, D5, D10 or D15.

The cell index (i.e. cell number) was monitored in real time every 15 minutes following treatment with C1, C5, C10, C15, D1, D5, D10 or D15 using an xCELLigence® real-time cell analysis (RTCA) assay platform. This instrument measures the cell index by monitoring electrical impedance in the wells containing the cells.

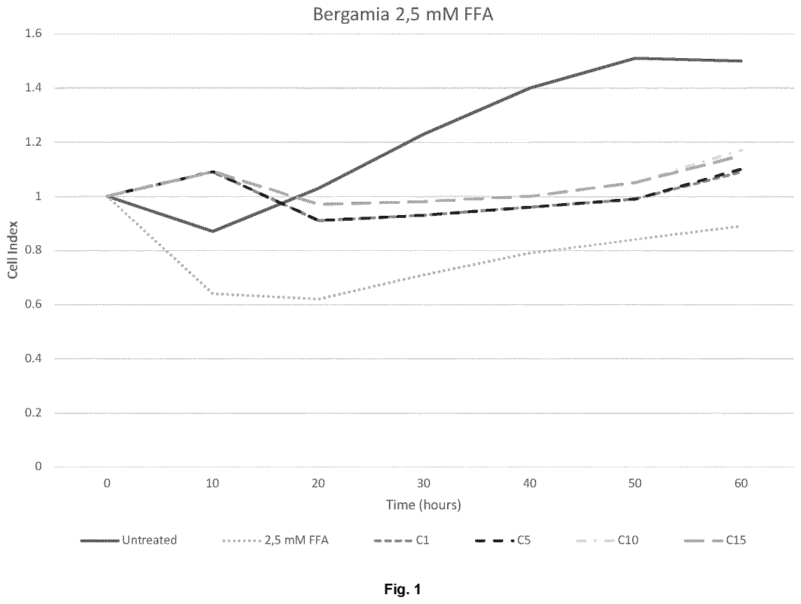

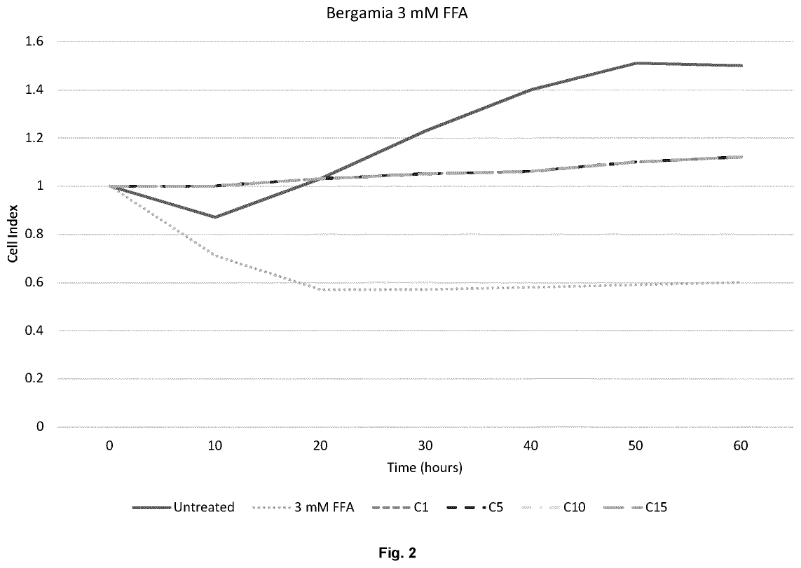

Treatment with the Citrus bergamia extract alone (C1, C5, C10 and C15 improves cell index relative to untreated control cells (FIGS. 1 and 2).

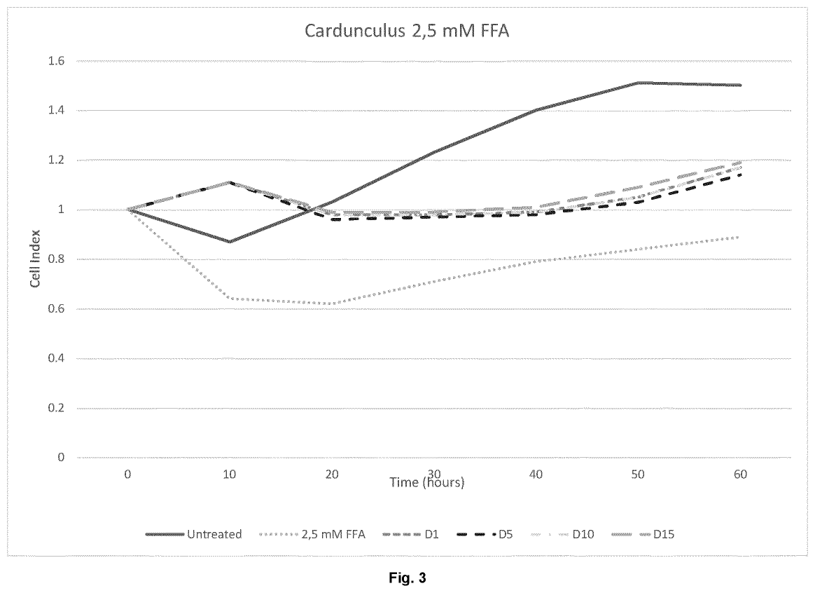

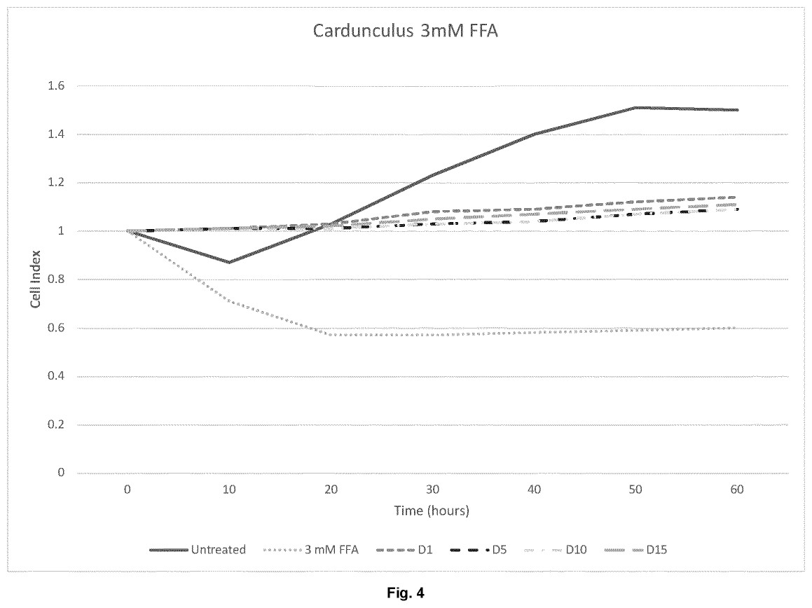

Treatment with the Cynara cardunculus extract alone (D1, D5, D10 and D15) also improves cell index relative to untreated control cells (FIGS. 3 and 4).

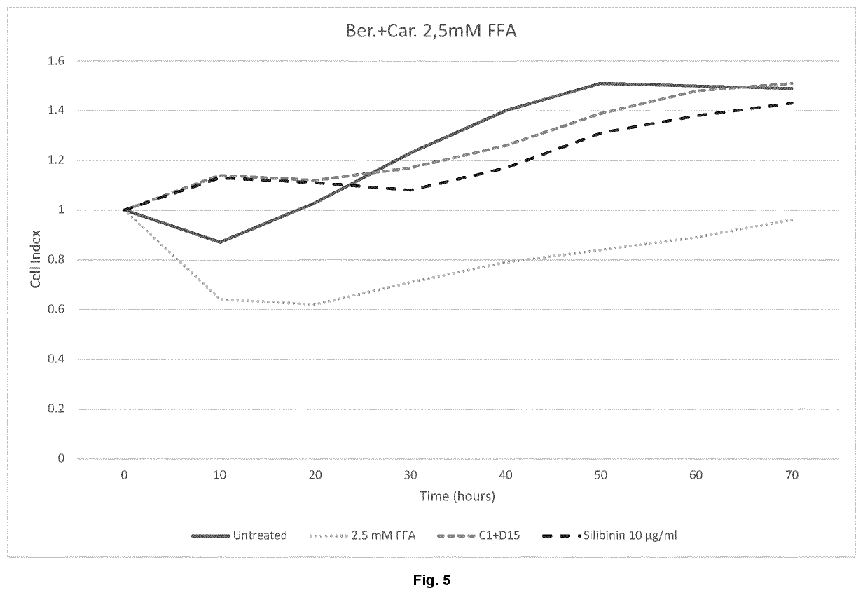

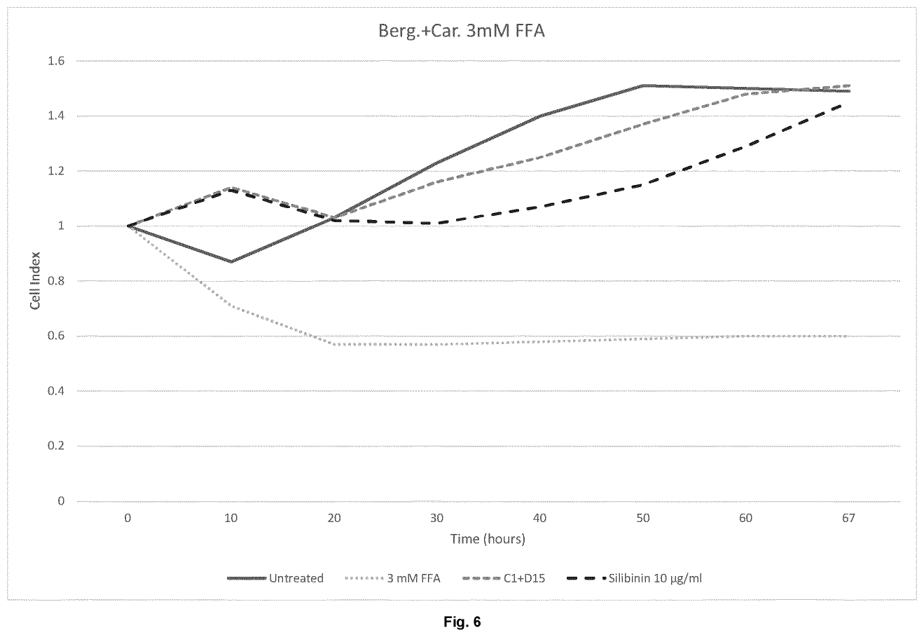

However, treatment with combination of both Citrus bergamia extract and Cynara cardunculus extract together improves cell index relative to control and treatment with each individual extracts (p <0.01) (FIGS. 5 and 6).

The improvement in cell index is similar to that provided by silibinin, a known treatment of hepatosteatosis (see Digestive and liver disease, 44, 2012, 334-342 and Translational Research, 159, 6, 2012). The combination provides a cell index equivalent to a cell sample untreated with fatty acids. A reduction of fat present in the liver is associated with beneficial metabolic and cardiovascular effects which in turn reduce the risk of metabolic and cardiovascular disease, and reduce build of cholesterol.

Lipid Content

The lipid content of the cells was measured by fixing the cells in formaldehyde (10%), staining with 0.21% Oil Red O isopropanol (Sigma-Aldrich, St. Louis, Mo., USA) for 10 minutes, and then washing with 60% isopropanol (Sigma-Aldrich). The accumulation of lipid droplets was examined using inverted microscope fluorescence with multi-channel LED lighting (Evos, Life technology, NY) measuring optic density (OD) at 490 nm.

The combination of Citrus bergamia extract and Cynara cardunculus extract reduces the content of lipid in hepatocytes caused by treatment with FFA 2.5 mM and 3.0 mM.

TABLE 1
Treatment with 2.5 mM FFA after 24 h

| | Relative |
|---|---|
| Conditions | levels of lipids |
| Control (untreated) | 100 |
| Following treatment with FFA 2.5 | 174 |
| Following treatment with FFA | 168 |
| 2.5 and then C5 | |
| Following treatment with FFA | 170 |
| 2.5 and then D15 | |
| Following treatment with | 157 |
| FFA 2.5 and then C5 + D15 | |

TABLE 2
Treatment with 3.0 mM FFA after 24 h

| | Relative |
|---|---|
| Conditions | levels of lipids |
| Control (untreated) | 100 |
| Following treatment with FFA 3.0 | 207 |
| Following treatment with | 198 |
| FFA 3.0 and then C5 | |
| Following treatment with | 202 |
| FFA 3.0 and then D15 | |
| Following treatment with | 188 |
| FFA 3.0 and then C5 + D15 | |

Expression of Fatty Acid Binding Protein 1 (FABP1)

Gene expression within the HepG2 liver cells was assessed by quantitative real-time polymerase chain reaction (qRT-PCR). Reverse transcription was carried out on 1 μg of total RNA using oligo (dT) primers and MultiScribe™ Reverse Transcriptase (Applied Biosystems, Milan, Italy), according to the vendors' instructions. Quantitative RT-PCR was performed in a 7900 HT Fast Start real-time PCR system (Applied Biosystems) in a mixture containing SYBR® Green PCR Master Mix (Life Technologies), specific primers, and 50 ng of cDNA in a total volume of 20 μL. The GAPDH housekeeping gene was used as a reference. The ΔCt protocol was used to determine the absolute values of gene expression.

Fatty acid binding protein (FABP1) is a gene that encodes the fatty acid binding protein found in liver. Fatty acid binding proteins (FABPs) are a family of small, highly conserved, cytoplasmic proteins that bind long-chain fatty acids and other hydrophobic ligands. FABP1 is known to be critical for fatty acid uptake and intracellular transport and also has an important role in regulating lipid metabolism and cellular signalling pathways.

FABP1 aids uptake of fatty acids into the cell and therefore lower expression of FABP1 is beneficial, because it is directly linked to the production of cholesterol transport proteins (Ipsen 2018).

Treatment with the Citrus bergamia extract alone (C15), Cynara cardunculus extract alone (D15) and their combination reduced expression of FABP1 (Table 3).

TABLE 3
gene expression of FABP1 after 72 hours

| | Relative expression |
|---|---|
| | of FABP1 (fold increase |
| Conditions | versus control) |
| Control (untreated) | 1 |
| Following treatment with FFA 3.0 | 5.417 |
| Following treatment with | 4.327 |
| FFA 3.0 and then C15 | |
| Following treatment with | 2.410 |
| FFA 3.0 and then D15 | |
| Following treatment with | 2.193 |
| FFA 3.0 and then C15 + D15 | |

Expression of Carnitine Palmitoyl Transferase (CPT2)

Carnitine palmitoyl transferase (CPT2) encodes a nuclear protein which is transported to the mitochondrial inner membrane. The encoded protein oxidizes long-chain fatty acids in the mitochondria. Defects in this gene are associated with mitochondrial long-chain fatty-acid (LCFA) oxidation disorders which in turn prevent effective metabolism of lipids.

CPT2 oxidises fatty acids and therefore higher expression of CPT2 is beneficial, because it avoids the hepatic accumulation of fatty acids, which in turn reduces the risk of the fatty acids being converted into cholesterol.

Treatment with the Citrus bergamia extract alone (C15), Cynara cardunculus extract alone (D15) and their combination increased expression of CPT2 (Table 4).

TABLE 4
gene expression of CPT2 after 72 hours

| | Relative expression |
|---|---|
| | of CPT2 (fold increase |
| Conditions | versus control) |
| Control (untreated) | 1 |
| Following treatment with FFA 3.0 | 0.2167 |
| Following treatment with | 0.3000 |
| FFA 3.0 and then C15 | |
| Following treatment with | 0.4133 |
| FFA 3.0 and then D15 | |
| Following treatment with | 0.7700 |
| FFA 3.0 and then C15 + D15 | |

Conclusion

The combination of Citrus bergamia extract and Cynara cardunculus extract reduces lipid content in hepatocytes caused by treatment with FFA 2.5 mM and 3.0 mM (Tables 1 and 2).

Treatment with the Citrus bergamia extract alone (C15) or the Cynara cardunculus extract alone (D15) reduced expression of FABP1, however treatment with the combination (C15+D15) provided a more pronounced reduction in the expression of FABP1 (Table 3).

Treatment with the Citrus bergamia extract alone (C15) or the Cynara cardunculus extract alone (D15) increased expression of CPT2, however treatment with the combination (C15+D15) provided a more pronounced increase in the expression of CPT2 (Table 4).

The combination of Citrus bergamia and Cynara cardunculus, produced an unexpected and very significant result compared to the single extracts in the in vitro model, in particular:
Reduced lipid accumulation in hepatocytes;
Reduced fatty acid accumulation through decreasing FABP1 expression; and
Increased the mitochondrial beta oxidation of fats through increasing CPT2 expression.

A consequence of these effects is that the liver can export less lipids by packaging them into water-soluble VLDL (very low density lipoprotein) particles. This in turn reduces the level of circulating cholesterol, which in turn prevents and treats dyslipidemia (particularly hypercholesterolemia) and reduces the likelihood of, cardiovascular and metabolic disease.

### Example 3: Formulations

General

The botanical extracts can be mixed with adequate excipients for the required dosage form. The botanical extracts can also be used for direct compression but are suitable also for dry or wet granulation, which is preferable in particular for sachets and stick packs.

The botanical extract/excipient blend (with or without a granulation step) can therefore be compressed with a rotary tablet-compressing machine equipped with suitable punches, encapsulated using a capsule filling machine, or filled into sachets or stick packs by an adequate packaging machine.

Tablet Formulation

A tablet composition containing the one or both of the extracts is prepared by mixing an appropriate amount of the extract with an appropriate diluent, disintegrant, compression agent and/or glidant. The compressed tablet may be film-coated.

The following tablet formulation was prepared:

| | % w/w | mg/tablet |
|---|---|---|
| Cynara cardunculus extract (5% chlorogenic acid) | 10 | 130 |
| Citrus bergamia extract (15% naringin) | 20 | 260 |
| Calcium phosphate | 50 | 650 |
| Microcrystalline cellulose | 14 | 182 |
| Maltodextrin | 4 | 52 |
| Silicon dioxide | 1 | 13 |
| Magnesium stearate | 1 | 13 |
| Total | 100 | 1300 |

Capsule Formulation

A capsule formulation is prepared by one or both of the extracts with an appropriate diluent and then filling the resulting mixture into standard hard gelatin capsules. An appropriate disintegrant and/or glidant can be included in appropriate amounts as required.

The following capsule formulation was prepared:

| | % w/w | mg/tablet |
|---|---|---|
| Cynara cardunculus extract (5% chlorogenic acid) | 20 | 130 |
| Citrus bergamia extract (15% naringin) | 40 | 260 |
| Maltodextrin | 38 | 244 |
| Silicon dioxide | 1 | 8 |
| Magnesium stearate | 1 | 8 |
| Total | 100 | 650 |

Granulate Formulation

A granulate formulation can be prepared by dry or wet granulation of one or both of the extracts with an appropriate diluent and then filling the resulting mixture into an appropriate dosage form, for example a sachet or stick pack.

The following granulate formulation was prepared:

| | % w/w | mg/tablet |
|---|---|---|
| Cynara cardunculus extract (5% chlorogenic acid) | 10.4 | 130 |
| Citrus bergamia extract (15% naringin) | 20.8 | 260 |
| D-mannitol | 60.4 | 755 |
| Hydroxypropyl cellulose | 4.4 | 55 |
| Flavouring | 2.48 | 31 |
| Sucralose | 0.08 | 1 |
| Silicon dioxide | 1.44 | 18 |
| Total | 100 | 1250 |

Percentage of Active Ingredient Components in Tablet, Capsule and Granulate Formulations

The exemplified tablet, capsule and granulate formulations each contain 130 mg of Cynara cardunculus extract and 260 mg of Citrus bergamia extract. In accordance with the composition of the extracts outlined in Example 1, this corresponds to the following amount of active ingredients in each extract:

| | | Amount of component |
|---|---|---|
| | | per dose |
| Extract (dose) | Component | of extract (% w/w) |
| Citrus bergamia (260 mg) | Naringin | 39 mg (15) |
| | Neoeritrocitrin | 26 mg (10) |
| | Neohesperidin | 39 mg (15) |
| Cynara cardunculus (130 mg) | Chlorogenic acid | 6.5 mg (5) |
| | Caffeoylquinic acid | 6.5 mg (5) |

| | | Amount of |
|---|---|---|
| | | component |
| | | per dose |
| | | of extract |
| Extract (dose) | Component | (% w/w) |
| Citrus bergamia (260 mg) | Naringin | 39 mg (15) |
| | Neoeritrocitrin | 26 mg (10) |
| | Neohesperidin | 39 mg (15) |
| | Melitidin | 7.8 mg (3) |
| | Brutieridin | 13 mg (5) |
| | Rutin | 0.3 mg (0.1) |
| Cynara cardunculus (130 mg) | Chlorogenic acid | 6.5 mg (5) |
| | Caffeoylquinic acid | 6.5 mg (5) |

1-15. (canceled)

16. A method for the treatment or prevention of a condition selected from the group consisting of dyslipidemia, cardiovascular disease, metabolic syndrome and hypercholesterolemia, the method comprising administering to a patient in need thereof a therapeutically effective amount of a combination of:
(i) naringin; and
(ii) chlorogenic acid.

17. The method of claim 16, wherein the combination further comprises neohesperidin.

18. The method of claim 16 or 17, wherein the combination further comprises neoeriocitrin, melitidin, brutieridin, or rutin, or combinations thereof.

19. The method of claim 16, wherein the weight ratio of naringin to chlorogenic acid is from 10:1 to 1:10.

20. The method of claim 16, wherein the weight ratio of naringin to chlorogenic acid is from 2:1 to 1:2.

21. The method of claim 16, wherein the naringin and the chlorogenic acid are administered separately, sequentially, or simultaneously.

22. The method of claim 16, wherein the naringin and the chlorogenic acid are administered simultaneously in a unitary formulation.

23. A method for the treatment or prevention of a condition selected from the group consisting of dyslipidemia, cardiovascular disease, metabolic syndrome and hypercholesterolemia, the method comprising administering to a patient in need thereof a therapeutically effective amount of a combination of:
(i) Citrus bergamia extract; and
(ii) Cynara cardunculus extract.

24. The method of claim 23, wherein the Citrus bergamia extract further comprises naringin.

25. The method of claim 23, wherein the Citrus bergamia extract comprises naringin, neoeriocitrin, and neohesperidin, or naringin, neoeriocitrin, neohesperidin and one or more compounds selected from melitidin, brutieridin or rutin.

26. The method of claim 23, claim 24, or claim 25, wherein the Cynara cardunculus extract comprises chlorogenic acid.

27. The method of claim 23, wherein the weight ratio of the Citrus bergamia extract to the Cynara cardunculus extract is from 10:1 to 1:10.

28. The method of claim 23, wherein the weight ratio of the Citrus bergamia extract to the Cynara cardunculus extract is from 2:1 to 1:2.

29. The method of claim 23, wherein the Citrus bergamia extract and the Cynara cardunculus extract are administered separately, sequentially or simultaneously.

30. The method of claim 23, wherein the Citrus bergamia extract and the Cynara cardunculus extract are administered simultaneously in a unitary formulation.
